(19) United States
(12) Patent Application Publication
(10) Pub. No.: US 2005/0245820 A1
(43) Pub. Date: Nov. 3, 2005

(54) Method and apparatus for verifying and correcting tracking of an anatomical structure during surgery

(76) Inventor: Vineet Kumar Sarin, Thousand Oaks, CA (US)

Correspondence Address: William L. Johnson, P.O. Box 1240, Somis, CA, 93066, US

(21) Appl. No.: 10/835,513

(22) Filed: Apr. 28, 2004

(51) Int. Cl. A61B 5/05

(52) U.S. Cl. 600/429

(57) ABSTRACT

A method and apparatus verifies and optionally, corrects tracking during computer assisted surgery by tracking a plurality of trackable targets, at least one of which is initially capable of positioning independent of the other targets. A geometric relationship between said targets is calculated and later verified, to detect instability of the trackable targets and their mounting system.

### BACKGROUND OF THE INVENTION

1. Field of the Invention

This invention relates to computer assisted surgery generally and more specifically to surgical navigation assisted by optically trackable targets, attachable to anatomical structures such as bone.

2. Description of the Related Art

Computer assisted tracking systems are increasingly finding employment in the medical field for tracking anatomical structures, for example during orthopedic surgery. Tracking systems are known which employ trackable targets in concert with a tracking or locating system. Such systems typically employ optical tracking, and allow a computer to directly acquire navigation information regarding a tracked structure such as a patient's femur, even as it is manipulated during a surgical procedure.

U.S. Pat. No. 5,828,770, for example, describes a system for determining the spatial position and angular orientation of an object in real-time. The disclosed system has a sensor section and a plurality of trackable markers, adapted for mounting to the object. The plurality of markers are activated simultaneously during each cycle of the sensor section after an initial marker-identification mode and energy emitted by such simultaneously activated markers is detected by the remotely located sensor. With such a system, because the markers have been each uniquely identified during the marker-identification mode, and the relative marker geometry is known a priori, the markers can be simultaneously activated, detected and tracked during a subsequent marker-tracking mode.

Any such tracking system is to some degree vulnerable to errors introduced by inadvertent slippage, movement, or deformation of the trackable markers and associated structure in relation to the anatomical structure to which they are attached. Such movement or deformation is difficult to detect during surgery, yet the accuracy of the tracking information from the markers is often critical to the successful outcome of a surgical procedure. Some practical method for verifying (and, preferably, correcting) the reliable fixation of the trackers is desired.

### SUMMARY OF THE INVENTION

In view of the above problems, the present invention provides a method and apparatus for verifying intra-operative stability of trackable surgical targets. The method is useful in connection with a computer assisted surgical navigation system that utilizes trackable targets coupled to an anatomical structure.

In accordance with the invention, a locating system acquires initial three-dimensional locations of a plurality of trackable targets. A computer then calculates an initial geometric relationship among the plurality of targets, based on the initial locations. At at least one later time, the locating system again acquires three-dimensional position information locating the same plurality of targets. The updated geometric relationship is then calculated and compared to the initial relationship. If the later relationship is not consistent with a non-deforming transformation of the initial relationship, a warning output is provided to alert the operator of tracker slippage or deformation. Motions of the trackers consistent with translation and/or rotation of a rigid body do not cause the warning output.

Optionally, in some embodiments of the invention a method of recalibration permits re-establishment of the correct target relationship, either by physically moving the targets or by recalculating tracker geometry, preferably based on redundant position information and assumptions regarding most likely target displacement.

These and other features and advantages of the invention will be apparent to those skilled in the art from the following detailed description of preferred embodiments, taken together with the accompanying drawings, in which:

### BRIEF DESCRIPTION OF THE DRAWINGS

FIG. 1 is a block diagram of a typical environment in which the invention operates, including patient and locating system;

FIG. 2 is a perspective view, partially exploded, showing an example of trackable targets and a mounting apparatus, in accordance with the invention, attached to a human bone;

FIG. 3 is a perspective view of one clamp suitable for attaching trackable targets to a bone;

FIG. 4 is a flow diagram showing steps of a procedure in accordance with the invention, for verifying reliable fixation of targets during surgery; and

FIG. 5 represents a two dimensional figure defined by trackable targets in accordance with the invention; together with a hypothetical alternate figure (dotted lines) which would be formed after a slippage or deformation of a target attachment mechanism; and

FIG. 6 is a perspective view of three trackers, with imaginary spheres visualized to illustrate geometric relationships involved in re-establishing proper position of a target after slippage has occurred.

### DETAILED DESCRIPTION OF THE INVENTION

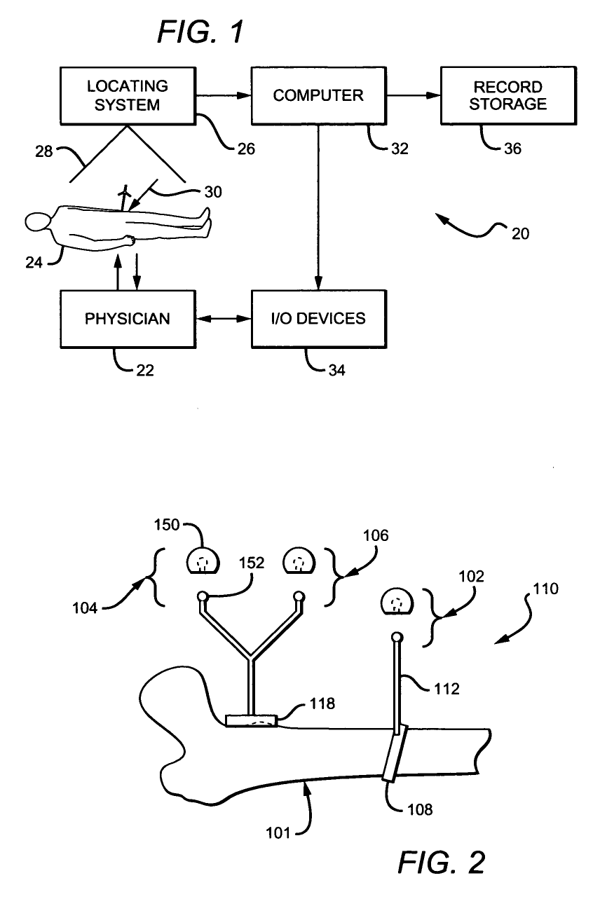

FIG. 1 shows a system-level block diagram of a system or apparatus 20 suitable for practicing the invention in a typical operating room environment. A physician or other professional 22 performs a joint replacement surgery (for example, total hip replacement) on a patient 24. An optical or equivalent locator or locating system 26 is disposed near the patient, so that the operating field is encompassed substantially within the field of view 28 of the locator 26. A suitable optical locator is available commercially, for example the “Polaris” available from Northern Digital Inc., in Waterloo, Ontario, Canada. Trackable targets 30 (preferably optical) are used during the operation, as more fully described below. The targets 30 allow the locator 26 to acquire the positions and orientations of tools and anatomical reference points, as described below.

The targets 30 could optionally be either active (for example, light emitting diodes) or passive (reflectors, for example). Similarly, the targets could be either wired or wireless without departing from the invention.

The locating system 26 is interfaced with and outputs tracking data to a digital computer 32, which interprets the optical tracking data as it is received. Using well known geometric relationships, the computer is programmed to deduce from the field of view the actual positions and orientations of the targets (and, by extension, the positions and orientations of any instruments and/or anatomical features that are in known relationship to the markers). For example, suitable optical targets utilizing single or multiple reflective spheres are available from Traxtal, Inc. in Toronto, Ontario, Canada. Targets with active light emitting devices such as LEDs are also available and could equivalently be used. Note that typical targets usually include two or more (non-collinear) components; this allows the locator and computer to determine the positions of the targets in three-dimensional space.

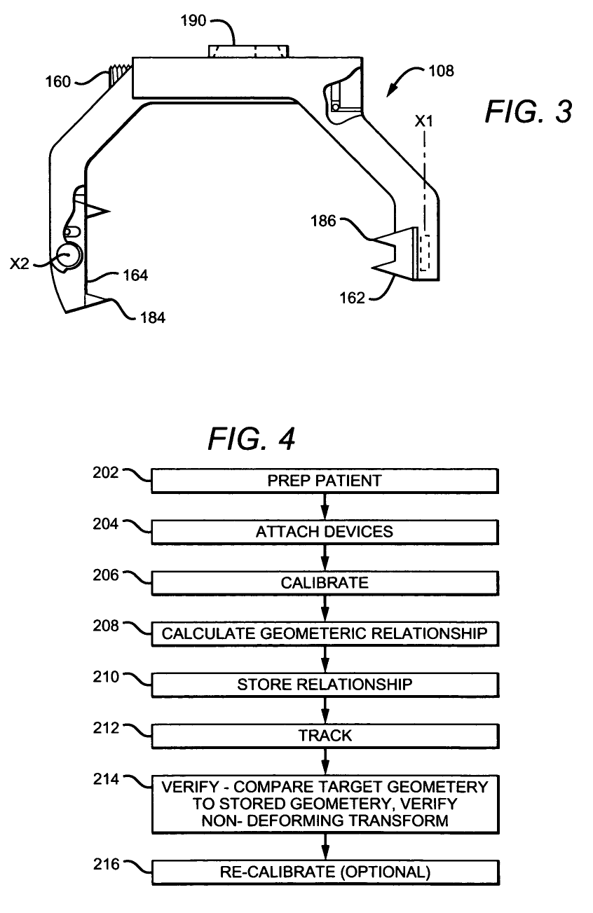

Preferably, the computer 32 is also programmed with a user-friendly interface (software) which facilitates the execution of the method of the invention (described below in connection with FIG. 3). The physician or other personnel can view output (for example on a video monitor) and input instructions to the computer 32 via I/O devices 34, which suitably could include a visual display (monitor), keyboard, printer, foot pedals, and other input/output devices such as conventional “mouse” or similar pointing devices. In addition to conventional system components, the apparatus of the invention should be programmed with a software, hardware, or firmware component that can calculate three-dimensional positions of trackable targets based upon raw image data acquired by the locating system.

Preferably, the system also includes a record storage device 36 such as a computer readable storage (magnetic, optical or other media), and/or simply a printer which prints out a summary of the operation and patient data for future reference or medical archiving.

FIG. 2 shows one suitable apparatus or set of devices for attaching trackable targets 30 to an anatomical structure. A human femur 101 is illustrated; however, the devices and method of the invention could be adapted to function on almost any rigid, substantially non-deformable structure. In accordance with the invention, at least one of the (optically) trackable targets, specifically, target 102, is attachable independently from (or at least moveable independently from) the other targets 104 and 106. A clamp or other moveable attachment device 108 is shown, and indeed such a moveable attachment device is preferred; however, in some embodiments other devices such as bone screws, wires, adhesives or other fixatives could be used for some or all of the targets.

It is extremely desirable, in accordance with the invention, that at least one of the attachable targets be independently moveable or capable of independent attachment in relation to the other targets, or otherwise capable of independent positioning: that is, it can be attached by a user at a position and in a geometric relationship that is not completely predetermined or fixed (in relation to the other targets). Thus, in the embodiment shown, the target 102 is attached via clamp 108 which can be positioned on the bone 101 in a variety or positions or orientations, as selected by the surgeon. Before tightening the clamp 108 the entire mechanism 110 (comprising 102, 108 and neck 112) are slidable and rotatable about the bone. After tightening the clamp mechanism 108, the mechanism 110 becomes fixed in relation to the bone, for at least some period of time.

Note that other mechanisms could be used to provide independence for at least one of the targets: for example, the targets could comprise reflective spheres mounted upon stems with moveable articulations, joints, or slidable or rotatable and lockable hinges.

It should be understood that more than one target may optionally be mounted on a single mounting mechanism such as 118. Furthermore, any number of total targets may be affixed to a common anatomical structure (such as bone), provided that the total number is at least two and preferably three or more. It is also significant that in accordance with the invention, the relationship between all the multiple targets need not be a priori known, stored, or predetermined, as in prior art tracking systems. At least one of the targets (and optionally, more than one) can be positioned by the surgeon almost arbitrarily (subject to certain minimal constraints, discussed below).

In accordance with the invention, one or more of the targets could optionally in some embodiments be attached by immobile means of fixation such as bone screws, provided that at least one target is capable of being positioned independently of the others. Each target could optionally be mounted to a separate clamp or bone screw; or some targets could share a clamp or screw mount. For example, in FIG. 2 the target 102 could be fixed to the bone, while 104 and 106 are mounted by a moveable clamp; on the other hand, 104 and 106 could be mounted to a fixed bone screw, provided that 108 is mounted moveably. Indeed, all of the targets could be mounted by separate bone screws provided that at least one of the targets is adapted to be mounted in a position which is not determined in advance by the positions of the other targets. Optionally, this freedom can be provided by mounting at least one target on a mounting device which is moveable in relation to others (for example, by a bendable, jointed, or articulated stem having the ability to retain its form after bending or adjustment)

FIG. 2 also shows, in partially exploded view, a particular trackable target which can be disassembled by removing reflective sphere 150 from mounting member 152. This type of detachable sphere mechanism facilitates, in some embodiments, the calibration step of the method described below.

The clamping devices such as 108 are preferred in one embodiment of the invention, to facilitate quick and easy fixation of the targets to a bony structure during surgery. One example of a suitable clamping device 108 is shown in FIG. 3. An adjustment screw 160 allows the clamp to be tightened and engage a bone. Moveable jaws 162 and 164 pivot about axes X1 and X2 to facilitate engagement of irregular, bony surfaces. Teeth 184 and 186 aid in securing a purchase on the bone. An attachment mechanism 190 is provided to permit mounting of one or more trackable targets on the clamp.

It will be apparent to those with skill in the art that variations on the clamp are possible to better interact with differing anatomical structures; the clamp shown in FIG. 3 is appropriate to attach to an upper femur at or near the greater trochanter, for example. Other clamps can be adapted to fit different bones, tissues, or even limbs. Although clamps are used in one embodiment of the invention, other fixing devices such as bone screws could be substituted in other embodiments. Independent means of fixation should be provided for at least one of the targets.

### Method of Verification

The method of verification includes general steps, as shown in FIG. 4. Each of the general steps includes further detailed steps which are described below in connection with FIG. 4.

As a preliminary step 202, the patient or subject is prepared and arranged in the field of view of the locating system 26. The anatomical structure to be tracked (in most instances, a bone such as the femur) is then identified and prepared for attachment of the trackable targets.

Once the anatomical structure is prepared, the surgeon attaches one or more clamps, screws, or other attachment devices capable of supporting trackable targets (Step 204). At least one of the targets, and optionally more than one, is attached and positioned independently of the others. The position of the targets is almost arbitrary, subject to certain constraints as follows: The targets should most preferably be placed in clear view of the locating system; they should preferably be placed in a non-collinear configuration; and they should preferably be placed in a configuration with broken symmetry. For example, with three targets it is most preferable to place the targets in a non-isosceles triangle. By implication, an equilateral triangle is not permitted, since equilateral is a special case of an isosceles triangle. Similarly, if more that three targets are used then symmetrical shapes such as square and rectangular configurations are to be avoided, since these yield ambiguous information to the locating system.

After or during the placement of the targets, the targets are identified by the locating system, preferably individually, in “calibration” step 206. To distinguish the targets, various methods can be employed. For example, the surgeon can touch an individual target with a trackable tool having previously known or fixed geometry, while cueing the computer to acquire the indicated position. Methods are known for optically tracking a probe with previously known geometry having optically trackable targets mounted thereon in a known, precisely predetermined configuration. To acquire the position of the tip of such a tool, it is a simple matter to locate the position and orientation of the targets, then calculate a translation to find the position of the probe tip, based on the previously known geometry of the tool. Alternatively, the surgeon can serially shadow or mask each target while cueing the computer (for example by foot pedal, in response to a menu prompt). This procedure allows the locating system and computer to associate optical inputs with individual targets to unambiguously calculate three-dimensional positions for each target.

During the “calibration” step 206 it is critical that the imaging apparatus (included in locating system 26) remain fixed in relation to the target being acquired. (Alternatively stated, the target must remain temporarily stationary in relation to the imaging apparatus during registration). This is not an onerous requirement, as the registration of each target can be performed in fractions of a second.

In one variation of the “calibration” step, the operator begins with the targets partially disassembled. Referring back to previous FIG. 2, in some embodiments the trackable targets comprise detachable reflective spheres which can be engaged with mounting members 152. Member 152 is preferably provided with an index depression or dimple to identify the point which will become the center of the reflective sphere 150 (once the sphere is mounted). In such an embodiment, calibration is performed by serially acquiring, with a trackable probe, the positions of the index depressions. The reflective spheres are then placed on the mounting members 152 after the centers have been acquired. Preferably, caution should be used to prevent the target or the mounting members from deforming or slipping while mounting the spheres to the mounting members 152.

Returning to the method of FIG. 4, once the targets are “acquired” (identified by the optical locating system and computer), the computer calculates (step 208) an initial geometric relationship among the targets and stores (210) this relationship in some form. In one embodiment employing three targets, the computer calculates a triangle ABC defined by the vertices (A,B, and C) at the positions of the three targets. The triangle relationship can be stored as three sides, as two sides and an angle, as three angles and a side, or in any form which uniquely defines the triangle in accordance with well known Euclidean geometry.

Although not strictly necessary for the verification method of the invention, it is convenient for tracking to use the acquired positions of three trackable targets to define a tracker coordinate system. This is done as follows: Given the positions of three points A,B, and C, we define A as origin. Next, we define the x-axis as vector from A to C (denoted vector AC). We then define a vector from A to B (denoted vector AB). We define the z-axis as AC×AB (cross product). Finally, we define y-axis as the “vector product” (also called “cross product”) of the z-axis with the x-axis. Other methods are possible. The method given in this paragraph provides orthogonal basis axes, which are useful to facilitate further calculation (for example in surgical navigation).

After the targets are acquired and the initial relationship stored, the surgeon is free to manipulate the anatomical structure. During manipulation the locating system and computer track (step 212) the three acquired targets and preferably provide feedback to the surgeon via a CRT or other output device. Tracking the targets includes refreshing from time to time the optical tracking data which locate the current positions of the trackable targets and their relationships to one another. Note that tracking the three targets does not necessarily uniquely locate the anatomical structure, unless further information is provided. If the relationship between the three targets and the anatomical structure is input to the computer, then the position of the anatomical structure can be inferred based upon the positions of the targets and the relationship between the targets and the anatomical structure. Optionally in some embodiments the position of an anatomical landmark could be input by other means such as a trackable touch probe or imagery. Once the position of the landmark is input, it can be tracked indirectly by tracking the targets and then correcting for the vector between the targets and the landmark (assuming a rigid relationship between the landmark and the targets). However, this tracking of anatomical landmarks is not necessary in all embodiments of the invention; in some embodiments only the targets are tracked. In such an embodiment the relationship between the targets' initial positions and later positions (relative positional data) is extremely useful for both navigation and verification.

First, the rotational position of the three targets is useful because it can allow a surgeon to return a bone or limb to a known reference position and/or orientation (for example, a “neutral” leg position). It can also aid a surgeon to move a structure while keeping it parallel to a desired reference orientation.

The relative information is further useful for tracking because it can compare an initial position with a later position. For example, an initial leg length or femoral offset can be directly compared with a later tracked leg length or offset to determine a surgical change in leg length or offset.

Consider a specific example, a common hip replacement procedure. After preparing the patient, but before dislocating the hip joint, the surgeon attaches the trackable targets as described above. During the surgery the locating system continues to track the femur. To check for changes (desired or undesired) in leg length and offset, after reduction of the joint the surgeon manipulates the femur while observing the CRT for feedback concerning the orientation of the femur. The computer informs the surgeon via the CRT when all of the requisite trackers are visible, and can provide visual feedback comparing the current leg position to a stored reference position (for example, a stored reference position having a previously defined relationship to the pelvis). The orientation of the trackers defines a rotational direction, which can be relied upon to re-orient the femur to a reference position. Furthermore, the tracked positions of the femoral targets allows calculation of changes in leg length and offset.

Finally, during or after navigation, the method of the invention includes the step (214) of verification. The tracking and verification steps 212 and 214 should most preferably be performed frequently, at short time intervals, to give nearly continuous verification of tracking stability. Such near-continuous, time-to-time verification gives the impression of real-time, continuous verification and thus provides a secure check on tracking during the procedure. Alternatively, the tracking and verification steps (212 and 214) could be performed at one or more discrete times, either under software control, hardware control, or even at the discretion of the operator. Such a variation may not give as great an impression of security, but may be adequate in some applications.

In step 214 the computer checks the geometric relationships among the trackable targets and compares them to the stored, initial relationship. If the trackable targets have not slipped or the apparatus has not been deformed during the procedure, then the relative geometry defined by the trackable targets should remain substantially constant (within acceptable margins of error). Acceptable margins of error could be user defined in advance of the procedure. For example only, in certain hip replacement surgeries using convenient target geometries, a margin of error of 2 millimeters movement for any given target sphere would typically be acceptable. Any deviation further than the acceptable margin should prompt an output (“alarm”) to the surgeon or other operator to inform of the discrepancy. This indicates that a problem occurred during the procedure. The surgeon is then left with the decision whether to modify or repeat the procedure, check accuracy of the procedure by other, independent means, or to attempt to re-establish the correct relationship between the trackable targets and the anatomical structure.

In some embodiments the optional step of “re-calibration” (step 216) is facilitated by the invention. Once the operator has determined, based on an alarm, that a target has slipped or other instability has occurred, the operator has the option to select “re-calibrate tracker” mode. In this mode the computer is controlled by software to aid the operator in re-establishing the correct target position. Based on the assumption that only one of the targets has moved (the most likely scenario, given independent targets), the operator attempts to manipulate the erroneous target back into correct position/orientation. The computer provides visual feedback, via the CRT display or other output device, to facilitate repositioning the target. Once the target is repositioned (within acceptable margin of error) the software resumes tracking as before (with time-to-time verification).

In an alternative method of “recalibration” the locating system and computer simply acquire and store a corrected, updated geometrical relationship between the targets, without physically moving the targets. The availability of this alternate method depends on the availability of redundant tracking information, for example, by use of more than three targets. Further details of the “re-calibration” step are discussed below, in connection with FIG. 6. In general, the “recalibration” step involves determining the most likely individual target movement which accounts for the deviation from the initially established relationship. After determining the most likely target movement, the computer can recalculate a new geometric relationship between the targets based on the new positions of the targets, which then becomes the reference relationship for future error detection.

The reliability of this verification method relies upon the independence of at least one of the trackable targets. Since the verification step checks only relative positions of the targets (relative to one another), any movement of the targets in concert—that is, as if attached to a rigid, non-deformable framework—would be indistinguishable from a translation or rotation of the anatomical structure to which the targets are mounted. However, with independently mountable targets precise movement in concert is so unlikely that the possibility may be neglected. Rather, if an error is to occur it most likely will involve slippage of only one target (or at worst, two targets in different directions by different amounts). Such errors cause a noticeable change in the relative positions of the targets, which is detected during the verification step.

After verification, the targets can be removed and the procedure completed in accordance with good medical practice.

The method as described would seem to require that the targets remain at all times during the procedure firmly attached to the mounting devices (clamps or other); but such fixation is not necessarily a strict requirement. In some embodiments the mounting instrumentalities can be provided with a releasable coupling, to allow temporary detachment of the targets from their mounts. Such releasable couplings can be provided between the bone clamp and stem portions of the target structures, for example. Such a releasable coupling (or couplings) still permit re-establishment of the verifiable relationship between targets, provided that the coupling is adapted to re-engage in a predictably repeatable manner, predictably re-establishing its original position and original orientation of the target vis-à-vis the bone. Such an embodiment is also within the scope of the invention. In some cases a releasable coupling may be advantageous to allow a surgeon freedom to perform awkward manipulations, unencumbered temporarily from the target array.

Compared with the conventional trackers, the invention offers greater flexibility in placing the trackable targets because the present invention does not require a specific, predetermined target geometry. This allows the operator to position the targets for his/her convenience (subject to certain constraints, identified above). The operator can dispose the targets toward the locator, notwithstanding the choice of position of the locating system. Thus, the angle of view is not as constrained by the available targets and clamp geometries. The operator also has the freedom to choose target positions which will not interfere with surgical tools or anatomical structures, or the manipulative patterns of the particular surgeons. As a simple example, consider the fact that many surgeons are left hand dominant and may prefer different tool and tracker positions as compared with a right handed surgeon. With the tracking and verifying method of the invention, different trackable targets are not required to accommodate the left handed surgeon.

More importantly, the method and apparatus of the invention prevent errors in surgical navigation by providing an alarm or other indication when a trackable target has slipped or bent. Prior tracking systems relied upon target systems in which individual targets were in rigid, predetermined relation with one another. If one target were to slip, in prior art systems all related targets would be certain to slip in concert. Thus, such systems offered no means of detecting inadvertent slippage or deformation of the target mount.

Calculations

The mathematical methods involved in registration and/or verification are relatively straightforward. In a particular embodiment, the registration essentially comprises calculating a triangle which is uniquely defined by three trackable targets, at least one of which is independently attached to the tracked anatomical structure.

Once the vertices are defined, they can be used according to simple construction rules to define a local coordinate system (associated with the tracked structure).

It should be understood that the methods discussed herein require three dimensional coordinate information locating the plurality of trackers. Such information can be derived, for example, by tracking reflective spheres with stereoscopic optical localizers. Such methods are known. Hardware and software to track individual targets are available, for example from Northern Digital Inc. in Ontario, Canada. The three dimensional positions of each individual target (active or passive) will be assumed as inputs in the calculations described herein.

We describe the calculations for a simple geometry involving three targets defining a non-isosceles triangle. More than three targets can be used, at the expense of some additional complexity. One way of extending the method to more than three trackers is to define multiple triangles, one for each triplet combination of targets. At least one of such triangles can be treated by the methods described in this disclosure.

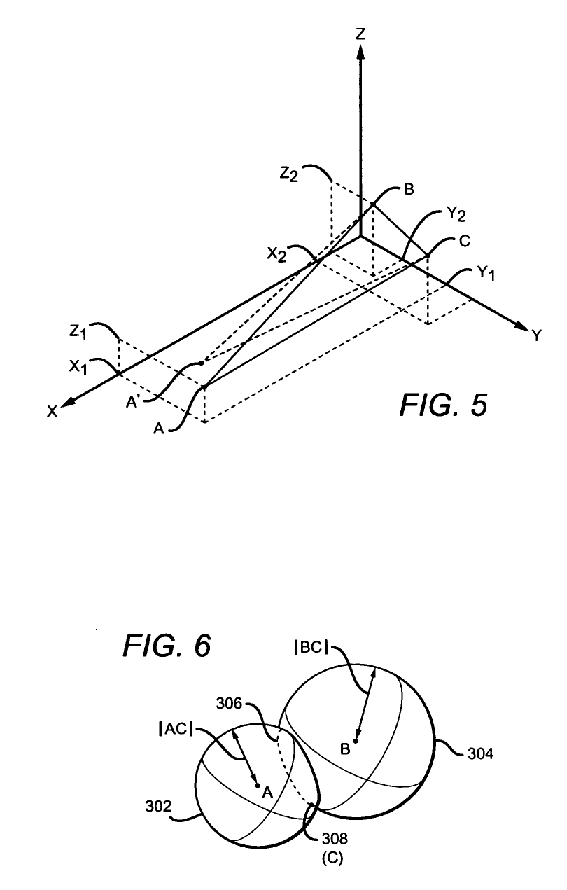

Referring now to FIG. 5, a triangle is defined by three vertices A, B, and C (corresponding to the tracked positions of three targets). Each vertex has three given coordinates in three space, thus A(x1, y1, z1), B(x2, y2, z2) and so forth. The vectors AB, BC, and CA are easily calculated by vector subtraction.

This allows calculation of the angles between BC and CA, and between CA and AB, by the corresponding equations. The magnitude (length) of each of the vectors AB, BC, and CA is also easily calculated by taking the square root of the dot product of the vector with itself.

These relationships allow calculation of parameters which define triangle ABC. Both angles and lengths of sides can be calculated by well known geometric principles. The complete set of lengths of sides and angles between sides is more than sufficient to uniquely characterize triangle ABC. In practice, it may be sufficient to store only a subset of those parameters which uniquely characterize the triangle. (For example, the lengths of all three sides and permutational order, or two angles and an included side).

During initial registration step, parameters which uniquely characterize the triangle ABC are calculated and stored.

FIG. 5 also shows in dotted lines a possible shifted configuration of markers ABC which might result if a target, say A, shifts to position A′ during surgery. Such a shift might result, for example, if the mounting device associated with target A slips during the procedure, or if the stem which mounts target A is inadvertently deformed (bent).

Recalculation during the verification phase will evidently produce different values for at least one of the angles or at least two of the side lengths for triangle A′BC as compared with the stored parameters associated with triangle ABC. This change in parameters serves as a basis to output a warning (such as “tracker error”) to a surgeon or other operator.

The above methods of calculation are simple but do not exhaust the possibilities for calculational methods which could be equivalently employed. The significant result that must be detected is whether the positions of the trackers at a later time differs from the original position by a deforming transformation. If the later locations of the targets differ from the original registration by mere translation and/or rotation, such a transformation is consistent with non-deforming motion of a rigid body (comprising the targets, mounting devices, and anatomical structure).

Optionally, additional output can be provided to give visual feedback to the surgeon relating real time position to an initial or stored position and orientation. A “bombsight” or cross-hair target, for example, can be useful to aid in returning a femur to an initial position or orientation (“native leg position” for example).

In a particular embodiment of the invention, the method is applied to track the femur of a patient during hip replacement surgery. As discussed above in connection with FIG. 4, during hip replacement surgery a plurality of trackable targets (such as reflective spheres or active LEDs) are attached via clamping or other fixing devices to a patient's femur, preferably before dislocation of the hip. At least one of the targets, and preferably more than one, are independently attached (via distinct clamping devices) and positioned for the surgeon's convenience. The targets need not be attached in accordance with a pre-determined unique geometry as in prior navigation systems. The native hip position is then acquired. Preferably, in this method additional trackable targets are used to independently track the pelvis. This allows the femur position to be dynamically transformed into a coordinate system rigidly associated with the pelvis. The targets are used for navigation during the hip replacement, preferably allowing the surgeon to monitor the relationship of the femur to the pelvis, and relating the real time position to the original “native” position.

During surgical manipulation (or even after reduction) the verification step (discussed above) allows the surgeon to verify that the targets have neither slipped nor deformed during the surgery, which often required forceful manipulations capable of bending or moving the targets. Verification is preferably performed from time to time, with frequency such that it appears to be continuous. In case of any deviation from initial tracker geometry, an alarm is activated, communicating the error to the surgeon. The chances are very remote that three or more targets would be inadvertently moved in parallel and by the same displacement in relation to the femur. Thus, the method of the invention provides an easy check of tracking accuracy, without requiring additional trackers, marking of bones, or radiographic imagery.

FIG. 6 shows typical geometry involved in the step of re-establishing proper tracker position (“recalibration,” not found in all embodiments). The actual geometry involved will depend greatly on the number of trackers and their relative positions. Thus, the figure shows merely an example which does not exhaust the possibilities. Given three trackable targets, A, B, and C, we assume for illustration that target C has been inadvertently moved (because, for example, its mounting clamp has slipped on the bone). The verification method has detected the error by the calculations and methods described above. Because of the stored geometrical relationships, we know that the correct position of the target C is at a point which is distance AC from point A; we further know that it is distance BC from point C. However, because the locating system and computer do not necessarily know the precise orientation of the anatomical structure (such as a bone, to which the targets are attached), we cannot calculate the proper point at which to relocate C without considering further constraints. We can calculate, however, that the proper position of C lies on the intersection of the spheres 302 and 304, having their centers at A and B and radii AC and BC, respectively. The intersections of these spheres define, most generally, a circle 306 (or in special cases, a point).

To find the point on the circle, additional constraints must be considered. In some cases, more than three targets (redundant targets) can be used to further define the proper recalibration position (to find point 308). In another method (also within the scope of the invention) we can take advantage of a further constraint imposed by the geometry of the mounting apparatus: for example, if we use a clamp as shown in FIG. 2, and a rigid stem of fixed length, the mount is constrained to only slide and rotate about the bone. Sliding and rotating the clamp can only achieve at most two points on the circle 306. Further constraint can be imposed by assuming that a small movement is more likely than a large movement (slippage).

Alternate methods can be used to constrain the permissible recalibration positions and thereby facilitate accurate re-establishment of tracking relationships. For example, the bone can be returned to an approximately known original position. The computer then offers visual guidance tools to assist the surgeon in returning all three (or more) targets to as near as possible to an original position and orientation. This method would work by removing the possibilities of extreme rotation or reorientation of the bone, leaving reduced ambiguity about the desired tracker position.

While several illustrative embodiments of the invention have been shown and described, numerous variations and alternate embodiments will occur to those skilled in the art. Such variations and alternate embodiments are contemplated, and can be made without departing from the spirit and scope of the invention as defined in the appended claims.

1. A method for verifying intra-operative stability of trackable surgical targets, useful in connection with a computer assisted surgical navigation system utilizing trackable targets coupled to an anatomical structure to dynamically track the structure during surgery, the method comprising:
acquiring initial three-dimensional locations of a plurality of trackable targets at an initial time;
calculating an initial geometric relationship among said targets based on the three-dimensional locations acquired, and without reference to predetermined parameters defining said geometric relationship;
storing data defining the initial geometric relationship;
acquiring later three-dimensional locations of said plurality of trackable targets;
calculating a later geometric relationship among said targets based upon the later acquired three-dimensional locations;
comparing said later geometric relationship with said initial geometric relationship;
determining whether said initial and said later geometric relationships are consistent; and
producing an output which indicates to a user the result of said determination, to indicate stability or instability of the attachment positions of said targets during some period of time.

2. The method of claim 1, comprising the further steps of:
before said initial acquisition, causing attachment of said plurality of trackable targets intra-operatively to a common, substantially non-deformable anatomical structure;
wherein at least one of said trackable targets is attachable independently of the others of said trackable targets.

3. The method of claim 2, wherein said trackable targets are attachable by removable clamps.

4. The method of claim 2, wherein said trackable targets comprise at least three targets.

5. The method of claim 4, wherein said geometric relationship comprises a polygon defined by a trackable target at each of its vertices.

6. A method of tracking a rigid anatomical structure, suitable for use in surgical navigation, comprising the steps of:
Attaching a plurality of trackable targets to a common anatomical structure, each target in fixed relation to said structure;
wherein at least one of said trackable targets is attachable independently from the remaining ones of said plurality of trackable targets;
and wherein the relationship among said trackable targets is not defined before attachment;
locating each of said plurality of trackable targets with a locating system to acquire an initial set of three-dimensional coordinates corresponding to each said target;
defining an initial geometric relationship among said trackable targets based on the acquired three-dimensional coordinates;
storing said initial geometric relationship; and
tracking said trackable targets from time to time to obtain time-varying three-dimensional coordinates corresponding to said targets, to obtain later positions of said targets.

7. The method of claim 6 comprising the further step of:
comparing said time varying three-dimensional coordinates to said initial set of three-dimensional coordinates of said trackable targets, and displaying a relationship between said later positions and said initial set of coordinates as an aid to surgical navigation.

8. A method of re-establishing registration of a computer assisted, surgical tracking system capable of tracking an anatomical structure with attached trackable targets, comprising the steps of:
detecting a deviation from an initially established spatial relationship among a plurality of trackable targets fixed to an anatomical structure; and
determining the most likely individual target movement which accounts for the deviation from said initially established relationship.

9. The method of claim 8, further comprising the steps:
based on the determined most likely individual target movement, relocating one of said targets to re-establish said initially established relationship.
providing visual feedback from a digital computer on a graphic display, to assist an operator in relocating one of said targets into said initially established relationship with the others of said plurality of targets.

10. The method of claim 9, wherein said step of relocating one of said targets comprises re-adjusting a clamp which fixes said target to the anatomical structure.

11. The method of claim 9, wherein said step of detecting a deviation comprises:
calculating an initial geometric relationship among said targets based on their tracked three-dimensional locations;
storing data defining the initial geometric relationship;
from time to time, acquiring updated three-dimensional locations for each of said plurality of targets;
calculating updated geometric relationship among said targets based on their updated three-dimensional locations; and
comparing said updated geometric relationship with said initial geometric relationship.

12. The method of claim 11, in which said initial and said updated geometric relationships comprise characteristics defining a polygon.

13. The method of claim 12, in which said polygon comprises a triangle.

14. The method of claim 8, further comprising the steps of:
storing said updated geometric relationship among said targets; and
Further tracking said anatomical structure based on said updated geometric relationship and the assumption of the most likely target movement to account for a change between said initial and said updated geometric relationships.

15. The method of claim 14, wherein said step of detecting a deviation comprises:
calculating an initial geometric relationship among said targets based on their tracked three-dimensional locations;
storing data defining the initial geometric relationship;
from time to time, acquiring updated three-dimensional locations for each of said plurality of targets;
calculating updated geometric relationship among said targets based on their updated three-dimensional locations; and
comparing said updated geometric relationship with said initial geometric relationship.

16. The method of claim 15, in which said initial and said updated geometric relationships comprise characteristics defining a polygon.

17. The method of claim 16, in which said polygon comprises a triangle.
